(19) United States
(12) Patent Application Publication
(10) Pub. No.: US 2006/0292227 A1
(43) Pub. Date: Dec. 28, 2006

(54) Extracellular matrix material particles and methods of preparation

(76) Inventor: Timothy B. McPherson, Edwardsville, IL (US)

Correspondence Address: BARNES & THORNBURG LLP, 11 SOUTH MERIDIAN, INDIANAPOLIS, IN, 46204, US

(21) Appl. No.: 11/472,947

(22) Filed: Jun. 22, 2006

Related U.S. Application Data
(60) Provisional application No. 60/693,379, filed on Jun. 23, 2005.

(51) Int. Cl. A61K 35/38; A61K 35/37

(52) U.S. Cl. 424/551

(57) ABSTRACT

A method for preparing a powdered extracellular matrix material is described. A particulate extracellular matrix material wherein at least 90% of the particles detectable by laser diffraction are about 420 microns or less. A particulate extracellular matrix material is also provided wherein at least 50% of the particles detectable by laser diffraction are about 210 microns or less.

### CROSS REFERENCE TO RELATED APPLICATIONS

This application claims priority under 35 U.S.C § 119(e) to U.S. Provisional Application Serial No. 60/693,379, filed on Jun. 23, 2005, incorporated herein by reference in its entirety.

### FIELD OF THE INVENTION

This invention relates to a method for preparing a powdered extracellular matrix material.

### BACKGROUND

Extracellular matrix material, such as submucosa tissue, has been shown to induce the growth of endogenous tissues when implanted as a graft construct into a host. Submucosa tissue grafts have been used successfully in vascular grafts, dermal grafts, urinary bladder grafts, and in hernia repair, and in replacement and repair of tendons and ligaments, and the like. A description of methods of preparation and various uses of submucosa tissues in graft constructs can be found in U.S. Pat. Nos. 4,902,508; 5,281,422; 5,275,826; 5,554,389; 6,793,939; 6,099,567; and other related U.S. patents. These submucosa tissue compositions have been shown to induce connective tissue remodeling and to promote wound healing, including cellular infiltration and neovascularization. Additionally, submucosa tissue graft constructs are resistant to infection and are non-immunogenic.

Submucosal extracellular matrix material may be obtained from the submucosa tissue of warm-blooded animals, for example, submucosa tissue may be obtained from pigs, cattle, or sheep or other warm-blooded vertebrates. Submucosa extracellular matrix material may be obtained from various sources including the small intestine, the urinary bladder, the stomach, or pericardial tissue, and the like.

Previous methods for creating submucosa tissue constructs have been limited with respect to the formation of three-dimensional shapes to use as scaffolds and did not result in the generation of the fine particulate extracellular matrix material described herein. Previous methods for producing a powdered extracellular matrix material have been unsuccessful in producing the fine particulate extracellular matrix material described herein because of the fibrous nature of collagen which tends to make the tissue curl and shred when it is comminuted, making the process of comminuting more difficult.

### SUMMARY

The methods of preparing the extracellular matrix material powder described herein result in particles that have a small size, and, thus, result in particulate extracellular matrix material (i.e., a powdered form of the extracellular matrix material) with a greater surface area for cell growth and remodeling. The methods and compositions described herein can be used for the formation of three- dimensional graft constructs for implantation, injection, or the powdered or particulate extracellular matrix material may be dispersed in a gel or ointment for topical use to induce the repair of damaged or diseased tissues in a host.

In one embodiment, a method is provided for preparing a powdered extracellular matrix material. The method comprises the steps of precipitating a crystalline material wherein the crystalline material is in a mixture with an extracellular matrix material, drying the extracellular matrix material in the mixture, and comminuting the dried extracellular matrix material into a powder form. In one embodiment, the crystalline material is impregnated or imbedded into the extracellular matrix.

In one embodiment of the method described in the preceding paragraph, the extracellular matrix material can comprise a submucosa tissue of a warm-blooded vertebrate. In another embodiment, the extracellular matrix material can be selected from the group consisting of small intestinal submucosa tissue, urinary bladder submucosa tissue, stomach submucosa tissue, and liver basement membrane tissue. In yet another embodiment, the crystalline material is selected from the group consisting of a salt and a sugar. In still another embodiment, the mixture can be obtained by adding a solution of the crystalline material to the extracellular matrix material, by adding the extracellular matrix material to a solution of the crystalline material, or by adding the crystalline material directly to a solution containing the extracellular matrix material. In other embodiments, the crystalline material can be selected from the group consisting of sodium chloride, potassium chloride, potassium phosphate, sodium phosphate, glucose, fructose, sucrose, lactose, and mannitol, and combinations thereof. In another illustrative embodiment, the concentration of the crystalline material in the mixture is about 5% w/v to about 50% w/v. In yet another illustrative embodiment, the crystalline material can be sodium chloride.

In the above-described method, the step of drying can be selected from the group consisting of freeze drying and air drying. In another embodiment, the method can further comprise the step of separating the crystalline material from the extracellular matrix material after the comminuting step. In another illustrative embodiment, the separating step can comprise washing the comminuted extracellular matrix material with water and centrifuging the washed material and removing the liquid from the extracellular matrix material powder. In another embodiment of the above-described method, the method can further comprise the step of re-comminuting the washed extracellular matrix material.

In yet another illustrative embodiment, the method can further comprise the step of mixing the powder form of the extracellular matrix material with a carrier to form an ointment composition. In the embodiment where the powder is used to form an ointment composition, the method can further comprise the step of compressing the powder form of the extracellular matrix material into a three- dimensional construct. In still another embodiment, the method can further comprise the step of adding a binding agent to the powdered form of the extracellular matrix material. In another embodiment, the binding agent can be selected from the group consisting of a fibrin glue and a collagen gel. In still another illustrative embodiment, the compressing step can be performed using a pre-formed mold.

In one embodiment, at least 50% of the particles of the particulate extracellular matrix material (i.e., powdered form of extracellular matrix material) detectable by laser diffraction are about 210 microns or less. In another embodiment, at least 90% of the particles of the particulate extracellular matrix material detectable by laser diffraction are about 420 microns or less.

In either of these embodiments, the particulate extracellular matrix material can be prepared from a submucosa tissue of a warm-blooded vertebrate. In another embodiment, either of the particulate extracellular matrix materials described in the preceding paragraph can be prepared from extracellular matrix material selected from the group consisting of small intestinal submucosa tissue, urinary bladder submucosa tissue, stomach submucosa tissue, and liver basement membrane tissue.

In yet another illustrative embodiment, either of the above-described particulate extracellular matrix materials can further comprise a carrier. In this illustrative embodiment, the carrier can be an oil or a gel. In either of the particulate extracellular matrix material embodiments, the particulate extracellular matrix material can be compressed into a three-dimensional construct. In this embodiment, the particulate extracellular matrix material can further comprise a binding agent. In this illustrative embodiment, the binding agent can be a fibrin glue or a collagen gel.

### BRIEF DESCRIPTION OF THE DRAWINGS

FIG. 1 shows Microtrac laser particle size analysis of small intestinal submucosa powder in purified water (sample TM081302A). The small intestinal submucosa powder was prepared according to the methods described herein.

FIG. 2 shows the Microtrac laser particle size analysis of small intestinal submucosa powder in purified water (sample TM081302B). The small intestinal submucosa powder was prepared according to the methods described herein.

### DETAILED DESCRIPTION

Methods are provided for preparing a powdered extracellular matrix material. This extracellular matrix material can be derived from submucosa tissue of a warm-blooded vertebrate, or, from other tissues. Exemplary tissues for use in the methods described herein are intestinal submucosa tissue, stomach submucosa tissue, urinary bladder submucosa tissue, and liver basement membrane tissue, and any other extracellular matrix tissue known in the art. The submucosa tissue can comprise the tunica submucosa delaminated from both the tunica muscularis and at least the luminal portion of the tunica mucosa of a warm-blooded vertebrate.

It is known that compositions comprising the tunica submucosa delaminated from both the tunica muscularis and at least the luminal portion of the tunica mucosa of the submucosal tissue of warm-blooded vertebrates can be used as tissue graft materials (see, for example, U.S. Pat. Nos. 4,902,508, 5,281,422, 4,956,178, and 5,554,389, incorporated herein by reference). Such submucosal tissue preparations are characterized by excellent mechanical properties, including high compliance, high tensile strength, and a high burst pressure point.

Other advantages of extracellular matrix material is its resistance to infection, stability, and lack of immunogenicity. Extracellular matrix material, such as intestinal submucosa tissue, has high infection resistance. Of course, appropriate sterilization techniques can be used to treat submucosal tissue. Furthermore, this tissue is not recognized by the host's immune system as “foreign” and is not rejected. It has been found that xenogeneic intestinal submucosa is not rejected following implantation as vascular grafts, ligaments, and tendons because of its composition (i.e., submucosal tissue is apparently similar among species). It has also been found that submucosal tissue has a long shelf-life and remains in good condition for at least two months at room temperature without any resultant loss in performance.

Extracelluar matrix materials, such as submucosal tissue, serve as a rapidly vascularized matrix for support and growth of new endogenous tissue. Thus, extracellular matrix materials, such as submucosal tissue, have been found to be trophic for host tissues with which they are attached or otherwise associated in their implanted environment. Extracellular matrix materials have been found to be remodeled (resorbed and replaced with autogenous differentiated tissue) to assume the characterizing features of the tissue(s) with which they are associated at the site of implantation or insertion. Additionally, the boundaries between the extracellular matrix materials and endogenous tissue are not readily discernible after remodeling. Common events to this remodeling process include widespread and rapid neovascularization, proliferation and granulation of mesenchymal cells, biodegradation of implanted extracellular matrix material, and lack of immune rejection.

Extracellular matrix materials are collagen based biodegradable matrices comprising highly conserved collagens, glycoproteins, proteoglycans, and glycosaminoglycans in their natural configuration and natural concentration. Such extracellular matrix materials may be used as a matrix for the regrowth of endogenous tissues. Extracellular matrix material, such as submucosal tissue can be obtained from various sources, for example, intestinal tissue can be harvested from animals raised for meat production, including, pigs, cattle and sheep or other warm-blooded vertebrates. Small intestinal submucosal tissue is a plentiful by-product of commercial meat production operations and is, thus, a low cost material.

In one embodiment, intestinal submucosal tissue typically comprises the tunica submucosa delaminated from both the tunica muscularis and at least the luminal portion of the tunica mucosa. In another embodiment, the intestinal submucosal tissue comprises the tunica submucosa and basilar portions of the tunica mucosa including the lamina muscularis mucosa and the stratum compactum which layers are known to vary in thickness and in definition dependent on the source vertebrate species.

The preparation of submucosal tissue is described in U.S. Pat. No. 4,902,508, the disclosure of which is expressly incorporated herein by reference. A segment of vertebrate intestine, for example, preferably harvested from porcine,bovine or bovine species, but not excluding other species, is subjected to abrasion using a longitudinal wiping motion to remove the outer layers, comprising smooth muscle tissues, and the innermost layer, i.e., the luminal portion of the tunica mucosa. The submucosal tissue is rinsed with saline and is optionally sterilized.

The extracellular matrix material derived from submucosal tissue can be sterilized using conventional sterilization techniques including glutaraldehyde tanning, formaldehyde tanning at acidic pH, propylene oxide or ethylene oxide treatment, gas plasma sterilization, gamma radiation, electron beam, and peracetic acid sterilization. Sterilization techniques which do not adversely affect the mechanical strength, structure, and biotropic properties of the submucosal tissue are preferred. Preferred sterilization techniques include exposing the submucosal tissue to peracetic acid, 1-4 Mrads gamma irradiation (more preferably 12.5 Mrads of gamma irradiation), ethylene oxide treatment or gas plasma sterilization.

In one embodiment, the source extracellular matrix material can be stored in a hydrated or dehydrated state. Lyophilized or air dried extracellular matrix material can be used as a source material for the extracellular matrix powder prepared by the methods described herein, without significant loss of its biotropic properties.

Methods of preparing other extracellular matrix materials for use in the methods described herein are known to those skilled in the art and may be similar to those described above for submucosal tissue. For example, see WO 01/45765 and U.S. Pat. Nos. 5,163,955, 5,281,422, 5,275,826, 5,554,389, 6,793,939, and 6,099,567 and U.S. patent application Ser. Nos. 09/691,345 and 09/691,590, each incorporated herein by reference. Extracellular matrix materials that can be used as a source material to prepare the extracellular matrix powder by the methods described herein include such tissue preparations as intestinal submucosa tissue, urinary bladder submucosa tissue, stomach submucosa tissue, liver basement membrane, pericardial tissue preparations, sheet-like collagen preparations, and the like.

Fluidized extracellular matrix material, such as intestinal submucosa tissue, has previously been successfully used to remodel damaged tissues. In one embodiment, fluidized or solubilized extracellular matrix material can be used in the methods described herein to produce powder forms of extracellular matrix material. Fluidized forms of extracellular matrix material are described, for example, in U.S. Pat. No. 5,275,826, incorporated herein by reference.

As used herein, “crystalline material” means a material that is capable of forming crystals, but does not mean a material that has necessarily crystalized.

A powdered extracellular matrix material can be prepared using the methods described herein. In one embodiment, the extracellular matrix material is mixed with a crystalline material. In another embodiment, the crystalline material precipitates in the mixture with the extracellular matrix material. In yet another embodiment, the crystalline material can be impregnated or imbedded into the extracellular matrix. In embodiments where the crystalline material is at a concentration in the mixture that is a supersaturating concentration, a saturating concentration, or a concentration that is about saturating, precipitation of the crystalline material can result. In illustrative aspects, the mixture can be obtained by adding a solution of the crystalline material to the extracellular matrix material or by adding the extracellular matrix material to a solution of the crystalline material or by adding the crystalline material directly to a solution containing the extracellular matrix material.

In still another illustrative embodiment, the crystalline material to be mixed with the extracellular matrix material can include any innocuous, soluble crystalline material. In another embodiment, the crystalline material can be a salt or a sugar. In another embodiment, the crystalline material can be selected from the group consisting of sodium chloride, potassium chloride, potassium phosphate, sodium phosphate, glucose, fructose, sucrose, lactose, and mannitol, or combinations thereof. In another embodiment, the concentration of the crystalline material in the mixture with the extracellular matrix material can be about 5% w/v to about 60% w/v, about 5% w/v to about 50% w/v, about 10% w/v to about 50% w/v, about 15% w/v to about 45% w/v, about 15% w/v to about 50% w/v, or about 20% w/v to about 50% w/v. In yet another embodiment, the concentration of the crystalline material in the mixture can be 30% w/v. In other embodiments, the crystalline material can be at a concentration that is saturating or supersaturating in the mixture.

In one embodiment, the extracellular matrix material can be mixed with the crystalline material for any period of time (e.g., about 5 seconds to about 96 hours), at any temperature, and with or without stirring.

In one embodiment, the extracellular matrix material in the mixture can be dried. In another embodiment, the step of drying the extracellular matrix material in the mixture can be performed by lyophilizing, freeze drying, air drying, drying with heat, or a combination thereof, or any other method that does not destroy the biotrophic properties of the extracellular matrix material. The extracellular matrix material can dried for any period of time (e.g., about 1 hour to about 72 hours) and at any temperature (e.g., about 4° C. to about 40° C.).

In another embodiment, the dried extracellular matrix material is comminuted into a powder form. In yet another embodiment, the dried extracellular matrix material can be comminuted in a dried, frozen state (e.g., frozen under liquid nitrogen and comminuted in a Waring blender). In one embodiment, the extracellular matrix material can be comminuted by tearing, cutting, grinding (e.g., using a pre- chilled mortar and pestle), fracturing by any means, or shearing (e.g., such as in a Waring blender, a Wiley knife mill, or an ultracentrifugal mill (using any screen size)), or otherwise producing a powdered form of the extracellular matrix material.

In one embodiment, combinations of methods of comminuting can be used. In one embodiment, pieces of extracellular matrix material can be comminuted by, for example, shearing in a high speed blender, or by grinding in a frozen or freeze-dried state to produce a powder.

Comminuting of the extracellular matrix material can be performed without loss of the material's apparent biotropic properties allowing use in methods such as injection or topical application to host tissues in need of repair. See U.S. Pat. No. 5,275,826. In one embodiment, the comminuting step can be performed at room temperature, but any useful temperature can be used. In another illustrative embodiment a carrier (e.g., dry sodium chloride, dry sucrose, liquid PEG-400, and the like) or combinations of carriers can be mixed with the extracellular matrix material before the comminuting step to aid in comminuting of the material.

In one illustrative embodiment discussed above, the step of drying the extracellular matrix material in the mixture can be performed by lyophilizing, freeze drying, air drying, drying with heat, or a combination thereof, or any other method that does not destroy the biotrophic properties of the extracellular matrix material. In the embodiments where the extracellular matrix material is dried or is dried and is comminuted into a powdered form, the crystalline material can be separated from the extracellular matrix material after the drying step or the comminuting step.

In another illustrative embodiment, the separating step can comprise washing the dried or dried and comminuted extracellular matrix material followed by addition of liquid, such as water, a centrifugation step, and removal of the liquid. In yet another embodiment, the dried extracellular matrix material can be washed with water or any other solvent in which the crystalline material can be dissolved to separate the crystalline material from the extracellular matrix material by addition of the solvent, a centrifugation step, and removal of the liquid. In another embodiment, dialysis can be used in place of washing to separate the crystalline material from the extracellular matrix material. In another embodiment, the extracellular matrix material can be re-comminuted after being washed and dried. In yet another illustrative aspect, the extracellular matrix material can be washed and dried and re- comminuted as many times as desired.

In any of the above-described embodiments, the drying of the extracellular matrix material can be repeated as many times as is necessary or is desired. In any of the above-described embodiments, the washing of the extracellular matrix material can be repeated as many times as is necessary or is desired. In any of the above-described embodiments, the comminuting of the extracellular matrix material can be repeated as many times as is necessary or is desired.

In one embodiment, the powdered extracellular matrix material can be mixed with a carrier to form an ointment composition. In another embodiment, the carrier can be selected from the group consisting of an oil or a gel. In another embodiment, the powdered extracellular matrix material can be compressed into a three-dimensional construct. In yet another embodiment, a binding agent may be added to the powdered extracellular matrix material wherein the binding agent can be selected from the group consisting of a fibrin glue and a collagen gel. In another embodiment, the powdered extracellular matrix material can be compressed by using a preformed mold.

In one of the illustrative embodiments described in the preceding paragraph, the powdered extracellular matrix material can be mixed with a carrier to form an ointment composition, such as for use in treating wounds, and the like. In one illustrative aspect, the carrier can include any suitable inert vehicle adapted to efficiently maintain the extracellular matrix material ingredients at the desired location such as gels, oils, creams, emulsions, and liquid preparations such as lotions. For example, the base of the carrier can be a fatty oil, a lanolin, a petroleum jelly, a paraffin, glycols, higher fatty acids and higher alcohols, organic and inorganic waxes, a vegetable oil, carboxymethyl cellulose, an aqueous base ointment, water in an oil emulsion, and the like.

In another embodiment described above, the powdered extracellular matrix material can be compressed into a three-dimensional construct. In this embodiment, a binding agent can be added to the powdered form of the extracellular matrix material. In one embodiment, the binding agent can be water soluble, and can have adhesive characteristics. In one illustrative embodiment, the binding agent can be any type of binding agent known in the art such as di-calcium phosphate, polyvinyl pyrrolidone, hydroxyethyl cellulose, hydroxypropyl cellulose, low molecular weight hydroxypropyl methylcellulose, polymethacrylate, or ethyl cellulose. In another embodiment, the binding agent can be selected from the group consisting of a fibrin glue and a collagen gel.

In another embodiment, the powdered extracellular matrix material can be compressed by using a preformed mold to make such constructs as, for example, graft constructs for plastic surgery applications, graft constructs for repair of bones or joints, or graft constructs for the repair of any other tissue (e.g., a cardiac valve) or organ for which molded, compressed constructs can be used.

In yet other illustrative embodiments, any of the extracellular matrix materials, described above can be impregnated with biological response modifiers such as glycoproteins, glycosaminoglycans, chondroitin compounds, laminin, poly-n- acetyl glucosamine, chitosan, chondroitin, growth factors, collagen, gelfoam, clotting agents or clot protectors, such as thrombin, fibrin, fibrinogen, anti-fibrinolytics, and the like, or combinations of these biological response modifiers. These biological response modifiers can be impregnated into the powder form of the extracellular matrix material or into the source extracellular matrix material before preparation of the powder.

As used herein the phrases “powdered extracellular matrix material,” “extracellular matrix material powder,” “powder form of the extracellular matrix material, “particulate extracellular matrix material,” and “particulate matrix material” have the same meaning.

In various illustrative embodiments, a particulate extracellular matrix material can be produced, using the methods described herein, wherein at least 40% of the particles detectable by laser diffraction are about 210 microns or less, wherein at least 45% of the particles detectable by laser diffraction are about 210 microns or less, wherein at least 50% of the particles detectable by laser diffraction are about 210 microns or less, wherein at least 60% of the particles detectable by laser diffraction are about 210 microns or less, wherein at least 70% of the particles detectable by laser diffraction are about 210 microns or less, wherein at least 85% of the particles detectable by laser diffraction are about 420 microns or less, wherein at least 90% of the particles detectable by laser diffraction are about 420 microns or less, wherein at least 93% of the particles detectable by laser diffraction are about 420 microns or less, wherein at least 95% of the particles detectable by laser diffraction are about 420 microns or less, or wherein at least 97% of the particles detectable by laser diffraction are about 420 microns or less. Any method known in the art to determine particle size can be used. The particles can be of any shape and can range from nanometers in size to millimeters in size.

Any of the above described illustrative method embodiments apply to the particulate extracellular matrix material (i.e., the powder form of extracellular matrix material). For example, as discussed above, in one embodiment, the particulate extracellular matrix material can be prepared from a submucosa tissue of a warm-blooded vertebrate. In another embodiment, the particulate extracellular matrix material can be selected from the group consisting of small intestinal submucosa tissue, urinary bladder submucosa tissue, stomach submucosa tissue, and liver basement membrane tissue.

In yet another embodiment, the particulate extracellular matrix material can further comprise a carrier. In another illustrative embodiment, the carrier can be any suitable inert vehicle adapted to efficiently maintain the active ingredients at the desired location such as gels, oils, creams, emulsions, and liquid preparations such as lotions. For example, the base of the carrier can be a fatty oil, a lanolin, a petroleum jelly, a paraffin, glycols, higher fatty acids and higher alcohols, organic and inorganic waxes, a vegetable oil, carboxymethyl cellulose, an aqueous base ointment, water in an oil emulsion, and the like.

In yet another illustrative embodiment, the particulate extracellular matrix material can be compressed into a three-dimensional construct. In a further illustrative embodiment, the particulate extracellular matrix material can further comprise a binding agent. In yet other illustrative embodiments, the binding agent can be any type of binding agent known in the art such as di-calcium phosphate, polyvinyl pyrrolidone, hydroxyethyl cellulose, hydroxypropyl cellulose, low molecular weight hydroxypropyl methylcellulose, polymethacrylate, or ethyl cellulose. In another embodiment, the binding agent can be selected from the group consisting of a fibrin glue and a collagen gel.

### EXAMPLE 1

### Preparation of an Extracellular Matrix Material Powder

The powdered extracellular matrix material mixture was made by using delaminated, small intestinal submucosa material prepared as described in U.S. Pat. No. 4,902,508, incorporated herein by reference, as a starting material. The delaminated, small intestinal submucosa material was pre-sterilized using peracetic acid. Strips of delaminated, small intestinal submucosa material were added to a 30% sodium chloride solution in distilled water. The mixture was stirred for 5 minutes at room temperature.

The small intestinal submucosa material strips were then removed from the 30% sodium chloride solution and were frozen by immersion in liquid nitrogen. The small intestinal submucosa material strips were dried in beakers in a lyophilizer. The dried extracellular matrix material strips were cut into small pieces with a pair of scissors, and were placed in an ultracentrifugal mill with a 5 mm screen. The sample was passed through the ultracentrifugal mill by centrifugation at 10,000 rpm.

A portion of the centrifuged sample was then placed in 100 ml of distilled water and was stirred to dissolve the sodium chloride. The sample was centrifuged at 4000 rpm for 10 minutes and the liquid was decanted. The washes were repeated two more times with centrifugation for 5 minutes at about 400 rpm. A final wash and centrifugation step was performed with centrifugation at 4000 rpm for 10 minutes. The resulting powder was lyophilized and was labeled sample TM081302A and was stored at −20° C. Particle size was determined by laser diffraction analysis as described below.

### EXAMPLE 2

### Preparation of an Extracellular Matrix Material Powder

A second powdered extracellular matrix material sample was prepared as described above (labeled sample TM081302B) except that, instead of an ultracentrifal mill, the lyophilized salt and extracellular matrix material mixture was passed through a Wiley knife mill (#60 screen). The resulting sample was washed as described above except that the sample was centrifuged two times for 5 minutes each at 500 rpm. The second sample was lyophilized and was labeled sample TM081302A and was stored at −20° C. The particle size was determined by laser diffraction analysis as described below.

### EXAMPLE 3

### Particle Size Analysis of an Extracellular Matrix Material powder

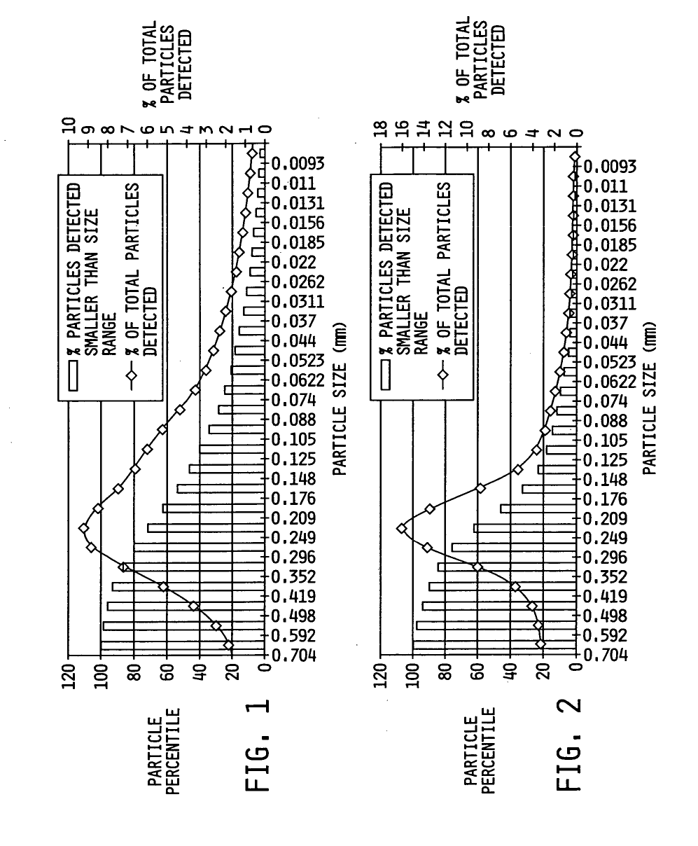

Samples of powdered, small intestinal submucosa tissue (samples TM081302A and TM081302B) were analyzed for particle size using a Microtrac laser particle size analyzer. Samples were thawed, were passed through a Wiley mill (#60 screen) as described above, and were dispersed in purified water. For analysis using a Microtrac particle analyzer, 704 microns is the largest size that the Microtrac analyzer is able to read. From the shape of the curves, the larger particles (i.e., larger than 704 microns) appear to represent about 1.5 to 4% of the total particles. As shown in FIGS. 1 and 2, sample TM081302B appears to have a narrower particle size distribution than sample TM081302A. Sample TM081302B also showed better flow properties than sample TM081302A. Approximately 60% of the particles for the samples ranged from about 0.352 to about 0.176 microns for the distribution of the particles that could be detected by the Microtrac analyzer. For both samples, at least 50% of the particles detectable by laser diffraction were about 210 microns or less and at least 90% of the particles detectable by laser diffraction were about 420 microns or less.

What is claimed is:

1. A method for preparing a powdered extracellular matrix material, the method comprising the steps of:
precipitating a crystalline material wherein the crystalline material is in a mixture with an extracellular matrix material; and
drying the extracellular matrix material in the mixture; and
comminuting the dried extracellular matrix material into a powder form.

2. The method of claim 1, wherein the extracellular matrix material comprises a submucosa tissue of a warm-blooded vertebrate.

3. The method of claim 1, wherein the extracellular matrix material is selected from the group consisting of small intestinal submucosa tissue, urinary bladder submucosa tissue, stomach submucosa tissue, and liver basement membrane tissue.

4. The method of claim 1, wherein the crystalline material is selected from the group consisting of a salt and a sugar.

5. The method of claim 1, wherein the mixture is obtained by adding a solution of the crystalline material to the extracellular matrix material, adding the extracellular matrix material to a solution of the crystalline material, or by adding the crystalline material directly to a solution containing the extracellular matrix material.

6. The method of claim 1, wherein the crystalline material is selected from the group consisting of sodium chloride, potassium chloride, potassium phosphate, sodium phosphate, glucose, fructose, sucrose, lactose, and mannitol, and combinations thereof.

7. The method of claim 6, wherein the concentration of the crystalline material in the mixture is about 5% w/v to about 50% w/v.

8. The method of claim 6, wherein the crystalline material is sodium chloride.

9. The method of claim 1, wherein the step of drying is selected from the group consisting of freeze drying and air drying.

10. The method of claim 1 further comprising the step of separating the crystalline material from the extracellular matrix material after the comminuting step.

11. The method of claim 10, wherein the separating step comprises washing the comminuted extracellular matrix material with water.

12. The method of claim 13, further comprising the step of re- comminuting the washed extracellular matrix material.

13. The method of claim 1, further comprising the step of mixing the powder form of the extracellular matrix material with a carrier to form an ointment composition.

14. The method of clam 1, further comprising the step of compressing the powder form of the extracellular matrix material into a three- dimensional construct.

15. The method of claim 14, further comprising the step of adding a binding agent to the powdered form of the extracellular matrix material.

16. The method of claim 15, wherein the binding agent is selected from the group consisting of a fibrin glue and a collagen gel.

17. The method of claim 14, wherein the compressing step is performed using a pre-formed mold.

18. A powder form of an extracellular matrix material prepared by the method of claim 1.

19. An ointment composition prepared by the method of claim 13.

20. A compressed extracellular matrix construct prepared by the method of claim 14.

21. A particulate extracellular matrix material wherein at least 50% of the particles detectable by laser diffraction are about 210 microns or less.

22. The particulate matrix material of claim 21, prepared from a submucosa tissue of a warm-blooded vertebrate.

23. The particulate matrix material of claim 21, wherein the extracellular matrix material is selected from the group consisting of small intestinal submucosa tissue, urinary bladder submucosa tissue, stomach submucosa tissue, and liver basement membrane tissue.

24. The particulate matrix material of claim 21, further comprising a carrier.

25. The particulate matrix material of claim 24, wherein the carrier is an oil or a gel.

26. The particulate matrix material of claim 21, compressed into a three-dimensional construct.

27. The particulate matrix material of claim 26, further comprising a binding agent.

28. The particulate matrix material of claim 27, wherein the binding agent is a fibrin glue or a collagen gel.

29. A particulate extracellular matrix material wherein at least 90% of the particles detectable by laser diffraction are about 420 microns or less.

30. The particulate matrix material of claim 29, prepared from a submucosa tissue of a warm-blooded vertebrate.

31. The particulate matrix material of claim 29, wherein the extracellular matrix material is selected from the group consisting of small intestinal submucosa tissue, urinary bladder submucosa tissue, stomach submucosa tissue, and liver basement membrane tissue.

32. The particulate matrix material of claim 29, further comprising a carrier.

33. The particulate matrix material of claim 32, wherein the carrier is an oil or a gel.

34. The particulate matrix material of claim 29, compressed into a three-dimensional construct.

35. The particulate matrix material of claim 34, further comprising a binding agent.

36. The particulate matrix material of claim 35, wherein the binding agent is a fibrin glue or a collagen gel.
